(19) United States
(12) Patent Application Publication
(10) Pub. No.: US 2025/0268814 A1
(43) Pub. Date: Aug. 28, 2025

(54) SIMULATED VERNIX COMPOSITION AND EMULSION INCLUDING THE SAME

(71) Applicant: Dermegen Inc., Hauppauge, NY (US)

(72) Inventors: Tianxiang Wang, Oviedo, FL (US); Rosa Dwyer, Bay Shore, NY (US); Paul Stettnisch, Lindenhurst, NY (US); Michael Ingrassia, Hauppauge, NY (US)

(73) Assignee: Dermegen Inc., Hauppauge, NY (US)

(21) Appl. No.: 19/059,864

(22) Filed: Feb. 21, 2025

Related U.S. Application Data
(60) Provisional application No. 63/556,758, filed on Feb. 22, 2024.

(51) Int. Cl. A61K 8/98; A61Q 19/00

(52) U.S. Cl. CPC A61K 8/982; A61Q 19/00; A61K 2800/5922; A61K 2800/81

(57) ABSTRACT

Described herein are compositions simulating the vernix cascosa. The compositions include in certain embodiments a squalane compound, a liquid monoester, and/or a vernix lipid. Also described herein are methods of preparing the compositions, and emulsions including the compositions.

### CROSS-REFERENCE TO RELATED APPLICATIONS

This application claims priority under 35 U.S.C. § 119(e) to U.S. Provisional Application No. 63/556,758, filed Feb. 22, 2024, which is hereby incorporated by reference in its entirety.

### BACKGROUND

Vernix cascosa is a lipid-rich material that covers the skin of the fetus and the newborn baby. Vernix cascosa includes wax and sterol esters, squalene, ceramides, cholesterol, triglycerides, and free sterols (Karkkainen et al., J Invest Dermatol, 44 (1965), pp. 333-338), as well as cellular elements (Agoratos et al., Am J Perinatol, 5 (1988), pp. 253-259). Vernix is considered either uniquely human (Pickens et al., J Invest Dermatol. 2000 November; 115 (5): 875-81) or existing in very few animal species other than human (Want et al., Scientific Reports 8, 7478 (2018)), although other structures, such as the periderm in rodents may play a similar part in utero (Wickett et al., J Invest Dermatol, 100 (1993), pp. 407-411; Okah et al., Pediatr Res, 37 (1995), pp. 402-408). Animals such as sheep produce lanolin, which also contains wax and sterol esters (Harris et al., Clinical and Basic Science Series, (2000), pp. 259-267). However, unlike vernix, this sebaceous secretion has not been reported to contain desquamated corneocytes.

In humans, vernix progressively coats the infant in a cephalocaudal manner during the last trimester of gestation. The high squalene and wax ester content in vernix strongly suggests that a significant portion of the lipid content is of sebaceous origin (Downing et al., J Invest Dermatol, 62 (1974), pp. 228-244). The onset of vernix production approximates the time of stratum corneum formation that begins anatomically in proximity to the pilosebaceous apparatus (Hashimoto, Arch Klin Exp Dermatol, 238 (1970), pp. 333-345; Hardman et al., J Invest Dermatol, 113 (1999), pp. 1106-1113).

Since vernix is a natural product for protecting the sensitive and fragile skins of human newborns, compositions similar to the vernix can be of interest for skin protection. For example, vernix-like compositions can be used for protecting the adult skin with damaged stratum corneum or poor skin barrier functions. The present invention addresses this need.

### SUMMARY

In one aspect, described herein is a composition comprising: a squalane compound, wherein the amount of the squalane compound ranges from 50 parts by weight to 95 parts by weight based on 100 parts by weight of the composition; a liquid monoester, wherein the amount of the liquid monoester ranges from 5 parts by weight to 30 parts by weight based on 100 parts by weight of the composition; and a vernix lipid, which comprises at least one selected from the group consisting of a cholesterol ester, a ceramide, a cholesterol, a phospholipid, a wax diester, a glycosphingolipid, and a cholesterol sulfate, and wherein the amount of the vernix lipid ranges from 0.05 parts by weight to 20 parts by weight based on 100 parts by weight of the composition, wherein the composition is a uniform oil phase mixture.

In another aspect, described herein is a method of preparing the composition, the method comprising: heating the squalane to an elevated temperature; adding the liquid monoester and the vernix lipid to the heated squalane to form a mixture while maintaining the elevated temperature; and cooling down the mixture to room temperature.

In another aspect, described herein is an emulsion, the emulsion comprising the composition, and water.

In another aspect, described herein is a composition comprising: a squalane compound, wherein the amount of the squalane compound ranges from 50 parts by weight to 95 parts by weight based on 100 parts by weight of the composition; a triglyceride, wherein the amount of the triglyceride ranges from 5 parts by weight to 30 parts by weight based on 100 parts by weight of the composition; and a vernix lipid, which comprises at least one selected from the group consisting of a cholesterol ester, a ceramide, a cholesterol, a phospholipid, a wax diester, a glycosphingolipid, and a cholesterol sulfate, and wherein the amount of the vernix lipid ranges from 0.05 parts by weight to 20 parts by weight based on 100 parts by weight of the composition, wherein the composition is a uniform oil phase mixture.

In another aspect, described herein are methods of supplementing naturally occurring lipids on the skin, comprising applying to the skin a composition according to certain embodiments, thereby forming a simulated vernix on the skin.

### BRIEF DESCRIPTION OF THE DRAWINGS

The following detailed description of exemplary embodiments will be better understood when read in conjunction with the appended drawings. For the purpose of illustrating, non-limiting embodiments are shown in the drawings. It should be understood, however, that the instant specification is not limited to the precise arrangements and instrumentalities of the embodiments shown in the drawings.

FIG. 1 compares the uniformity of some non-limiting examples of compositions according to the present invention (Formulae 1 and 2) with that of a similar composition (Formula 3), in accordance with some embodiments.

FIG. 2 presents corneometer data showing results of a skin hydration test using a composition in accordance with some embodiments.

FIG. 3 presents vapometer data showing results of a transepidermal water less (TEWL) test using a composition in accordance with some embodiments. “↓” lower than the basic data (P<0.05). “***” shows that there is difference on statistics.

FIG. 4 are photos of the same area of sensitive skin, following irritation and assessed at 0 weeks (left) and 2 weeks (right) of treatment with a composition in accordance with some embodiments.

### DETAILED DESCRIPTION

The following disclosure provides many different embodiments, or examples, for implementing different features of the provided subject matter. Specific examples of components and arrangements are described below to simplify the present disclosure. These are, of course, merely examples and are not intended to be limiting. For example, the formation of a first feature over or on a second feature in the description that follows may include embodiments in which the first and second features are formed in direct contact, and may also include embodiments in which additional features may be formed between the first and second features, such that the first and second features may not be in direct contact. In addition, the present disclosure may repeat reference numerals and/or letters in the various examples. This repetition is for the purpose of simplicity and clarity and does not in itself dictate a relationship between the various embodiments and/or configurations discussed.

Compositions simulating natural vernix as well as cosmetics using the same have been proposed. For example, U.S. Pat. No. 7,807,188 B2 describes such compositions. However, this patent does not describe compatibility with a lipid matrix, which could negatively impact the performance of the simulated vernix.

Squalene is a component of vernix and is produced by the human body naturally. Squalene is considered to have many benefits: the compound has been used to fight cancer and as an antioxidant, and is known to detoxify, hydrate and possesses emollient activities. The ability of the human body to produce squalene naturally, however; declines as individuals age. As such, it is desirable to administer squalene topically to humans, such as adult humans. However, squalene is unstable as the molecule include multiple double bonds which undergo oxidization upon contacting oxygen in the air.

Squalane is a saturation product of squalene. Squalane is stable and possesses many desirable features of squalene, has excellent compatibility with human bodies, is a good moisturizer for the hair, skin and nails, and is considered a potent anti-aging moisturizer.

As such, in a non-limiting aspect, the study as described in the instant specification (“the present study”) aimed to develop compositions simulating vernix which use squalane as the carrier to provide skin barrier function improvement and host defense functions to the skin. The present study discovered that squalane is not a good solvent for various components of vernix as some vernix components, such as ceramides, do not have good solubility in squalane. The present study thus aimed to resolve the compatibility issues between squalane and the vernix components by including additional solubilizers, such as polysorbates, safflower oleosomes, propanediol, PEG hydrogenated castor oil, which have been used in the art to solubilize hydrophobic oils into water. Unfortunately, the above solubilizers did not improve the solubility of the vernix components in squalane.

Surprisingly, the present study discovered that a class of liquid monoesters are able to provide synergistic effect as co-solubilizers and allowed the solubilization of vernix components such as ceramides, cholesterol, sterol esters, and phospholipids, in squalane.

Accordingly, in some aspects, the present invention is directed to a simulated vernix composition. In some embodiments, the simulated vernix composition form a uniform oil phase.

In some aspects, the present invention is directed to an emulsion including an oil phase simulated vernix composition and water.

In some aspects, the present invention is directed to a skin care composition including a simulated vernix composition or the emulsion comprising the same.

### Definitions

As used herein, each of the following terms has the meaning associated with it in this section. Unless defined otherwise, all technical and scientific terms used herein generally have the same meaning as commonly understood by one of ordinary skill in the art to which this disclosure belongs. Generally, the nomenclature used herein and the laboratory procedures in animal pharmacology, pharmaceutical science, peptide chemistry, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.”

“About” as used herein when referring to a measurable value such as an amount, a temporal duration, and the like, is meant to encompass variations of +20% or +10%, in certain embodiments ±5%, in certain embodiments ±1%, in certain embodiments ±0.1% from the specified value, as such variations are appropriate to perform the disclosed methods.

“Squalane compound,” as used herein, refers to squalane or any other farnesene derivatives. In some embodiments, the squalane compound is derived from a vegetable source such as sugarcane (e.g., Neossance, Amyris), sugar beet, wheat, or the like; a fruit sources such as olive or the like; an animal source such as shark or the like; or another source.

“Farnesene,” as used herein, refers to a set of six closely related chemical compounds which all are sesquiterpenes with a chemical formula of C₁₅H₂₄. Farnesene is the building block for many functional ingredients for topical administration and/or cosmetic compositions. For example, hydrogenation and polymerization of two farnesenes produces squalane with the chemical formula C₃₀H₆₂. One or more farnesenes may be hydrogenated, polymerized, carboxylated, esterified, or, added with a hydroxy functional group, and so forth. These farnesene derivatives can be further used as building blocks to make other derivatives. These derivatives can be emollients, thickeners, emulsifiers, surfactants, amino surfactants, wax, glossy agents, and the like.

The term “liquid monoester,” as used herein, refers to an ester that is liquid at room temperature and atmospheric pressure (25° C., 1 atm) and which comprises in the structure at least one hydrocarbon-based chain containing 2-45 carbon atoms. The liquid monoesters are the esters of alcohol and polyols with monocarboxylic or polycarboxylic acids, at least one of the alcohol or polyols and/or acids comprising at least one hydrocarbon-based chain containing at least 6 carbon atoms. In some embodiments, the liquid fatty ester is chosen from esters of a fatty acid (at least 6 carbon atoms) and of a monoalcohol, more particularly from esters of a fatty monoacid and of a monoalcohol. In some embodiments, the alcohol and/or the acid are saturated. In some embodiments, both the alcohol the acid for forming the liquid monoester are saturated. In some embodiments, the liquid fatty ester has the formula R¹—COOR², in which: R¹ is a linear or branched, saturated or unsaturated, optionally mono- or polyhydroxylated hydrocarbon-based radical, containing from 2 to 45 carbon atoms, preferably containing from 7 to 30 carbon atoms, and R² is a linear or branched, saturated or unsaturated, optionally mono- or polyhydroxylated hydrocarbon-based radical, containing from 1 to 20 carbon atoms. In some embodiments, R¹ is a linear or branched alkyl (saturated) radical containing 7 to 21 carbon atoms, such as from 8 to 17 carbon atoms, such as from 8 to 15 carbon atoms. In some embodiments, R¹ or R² is a farnesene group or farnesene derivative group.

The term “triglyceride,” as used herein, refers to a triacylglycerol compound,

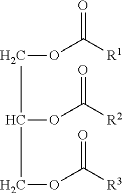

where R¹, R², and R³ are each independently C₆-C₃₀ saturated or unsaturated linear hydrocarbon chains, optionally substituted with one or more hydroxyl groups. Triglycerides are triesters of glycerol where the acyl groups are derived from fatty acids. Non-limiting examples of include esters derived from stearic acid, oleic acid, palmitic acid, and alpha-linolenic acid. Triglycerides can have three of the same acyl group, two of the same acyl group and one different, or all three different. Triglycerides can be found in nature, e.g., as animal fats or vegetable fats. Non-limiting examples of vegetable fats that are primarily composed of triglycerides include olive oil and shea butter.

The term “fatty acid,” as used herein, refers to a compound of the formula R—CO₂H, where R is a C₆-C₃₀ saturated or unsaturated linear hydrocarbon chains, optionally substituted with one or more hydroxyl groups. Non-limiting examples of fatty acids include stearic acid, oleic acid, palmitic acid, and alpha-linolenic acid.

The term “fatty alcohol,” as used herein, refers to a compound of the formula R—OH, where R is a C₆-C₃₀ saturated or unsaturated linear hydrocarbon chains, optionally substituted with one or more hydroxyl groups. Non-limiting examples of fatty alcohols include lauryl alcohol, myristyl alcohol, stearyl alcohol, oleyl alcohol, and behenyl alcohol.

The term “lipid,” as used herein, is defined in accordance with Fahy et al. (“Update of the LIPID MAPS comprehensive classification system for lipids,” J Lipid Res. 2009 April; 50(Suppl): S9-S14; the entirety of the reference is hereby incorporated by reference). Non-limiting examples of lipids include, but are not limited to fatty acids, glycerolipids, glycerophospholipids, sphingolipids, sterol lipids, prenol lipids, saccharolipids, and polyketides.

The term “simulated vernix,” as used herein, refers to a non-naturally occurring layer of lipid-rich material that simulates the properties and effects of naturally occurring vernix caseosa. In some embodiments, a composition (i.e., a simulated vernix composition as described in the following section(s)) can be applied to the skin and in doing so supplement the naturally occurring lipids present on the skin. Applying the composition to the skin thereby forms a simulated vernix on the skin.

The term “vernix lipid,” as used herein, refers to lipids found in vernix, or lipids that belong to the same category as the lipids found in vernix. In some embodiments, the term refers to the lipids that are found in the vernix, and are in lower concentration or do not exist in the skin surface lipid of adult skin or stratum corneum, or lipids belonging to the same category. In some embodiments, the vernix lipid includes the lipids whose levels decreases as the skin ages or become sensitive skin. The lipid compositions of vernix, skin surface and stratum corneum are described in, for example, Sumida et al. (“Studies on the function of Vernix caseosa: The secrecy of Baby's skin.” Cannes, France: International Federation of the Societies of Cosmetic Chemists 20th International Conference; 1998. pp. 1-7), and Hoath (2003) (Neonatal skin: structure and function (2. ed., rev. and expanded. ed.). New York [u.a.]: Dekker. pp. 193-208. ISBN 0-8247-0887-3), the entireties of which are hereby incorporated by reference, and are summarized below in Table 1:

TABLE 1
Comparison of the Lipid Compositions between Vernix Caseosa
and Adult Skin Surface Lipids (Weight Percentage)

| Lipid Categories | Vernix caseosa | Skin surface lipids |
|---|---|---|
| Cholesterol esters | 30.6 | 3 |
| Ceramides | 17.9 | — |
| Triglycerides | 15.1 | 41.8 |
| Cholesterol | 7.5 | — |
| Free fatty acids | 6.5 | 18.4 |
| Phospholipids | 6.1 | 1.5 |
| Wax esters | 6 | 20.3 |
| Wax diesters | 3.7 | — |
| Glycosphingolipids | 2.4 | — |
| Cholesterol sulfate | 0.3 | — |
| Alkanes | — | 2.8 |

### Simulated Vernix Composition

In some aspects, the present invention is directed to a simulated vernix composition. In some embodiments, the simulated vernix composition includes a squalane compound; a liquid monoester; and a vernix lipid. In other embodiments, the simulated vernix composition includes a squalane compound; a triglyceride; and a vernix lipid.

In some embodiments, the squalane compound includes squalane. In some embodiments, the squalane compound consists of squalane.

In some embodiments, an amount of the squalane compound ranges from about 50 parts by weight to about 90 parts by weight, such as about 55 parts by weight to about 90 parts by weight, about 60 parts by weight to about 90 parts by weight, about 65 parts by weight to about 90 parts by weight, about 70 parts by weight to about 90 parts by weight, or about 75 parts by weight to about 90 parts by weight, based on 100 parts by weight of the simulated vernix composition.

In some embodiments, the liquid monoester includes jojoba oil, or a polyglyceryl isostearate (such as polyglyceryl-4 isostearate or polyglyceryl-2 isostearate). In some embodiments, the liquid monoester consists of jojoba oil. Jojoba oil, sometimes referred to as “liquid wax,” is composed almost entirely (˜97%) of mono-esters of long-chain fatty acids. In some embodiments, the liquid monoester consists of the polyglyceryl isostearate.

In some embodiments, the liquid monoester includes a glyceryl or polyglyceryl monoester, a xylitol monoester, a sorbitan monoester, or combinations thereof. In some embodiments, the liquid monoester consists of the glyceryl or polyglyceryl monoester, the xylitol monoester, or the sorbitan monoester.

In some embodiments, an amount of the liquid monoester ranges from about 5 parts by weight to about 40 parts by weight, such as about 7.5 parts by weight to about 35 parts by weight, about 10 parts by weight to about 30 parts by weight or about 15 parts by weight to about 25 parts by weight based on 100 parts by weight of the simulated vernix composition.

In some embodiments, an amount of the triglyceride ranges from about 5 parts by weight to about 40 parts by weight, such as about 7.5 parts by weight to about 35 parts by weight, about 10 parts by weight to about 30 parts by weight or about 15 parts by weight to about 25 parts by weight based on 100 parts by weight of the simulated vernix composition. In some embodiments, the triglyceride includes shea butter.

In some embodiments, the vernix lipid includes a cholesterol ester, a ceramide, a triglyceride, a cholesterol, a free fatty acid, a phospholipid, a wax ester, a wax diester, a glycosphingolipid, a cholesterol sulfate, an alkane, or combinations thereof. In some embodiments, the vernix lipid includes a cholesterol ester, a ceramide, a cholesterol, a phospholipid, a wax diester, a glycosphingolipid, a cholesterol sulfate, or combinations thereof. In some embodiments, the vernix lipid includes a ceramide, as well as one or more additional components listed above.

In some embodiments, an amount of the vernix lipid ranges from about 0.05 parts by weight to about 30 parts by weight, such as about 0.5 part by weight to about 27.5 parts by weight, about 1 part by weight to about 25 parts by weight, about 2 parts by weight to about 20 parts by weight, about 5 parts by weight to about 17.5 parts by weight, about 7.5 parts by weight to about 15 parts by weight, based on 100 parts by weight of the simulated vernix composition.

In some embodiments, the vernix lipid includes a cholesterol ester. In some embodiments, the cholesterol ester includes a cholesterol esterified to a fatty acid having a chain length ranging from 10 to 24 (either saturated or unsaturated fatty acid). Non-limiting examples of cholesterol esters include cholesteryl: acetate, behenate, 11-hydroperox-eicosatetraenoate, ferulate, henicosenoate, heptadecanoate, icosapentaenoate, icosatetraenoate, icosatrienoate, laurate, myristate, nonadecanoate, nonadecenoate, octadecadienoate, octadecatrienoate, octadecenoate, palmitate, pentadecanoate, stearate, tetracosadienoate, and the like. In some embodiments, an amount of the cholesterol ester ranges from 0.05 parts by weight to about 15 parts by weight, such as about 0.1 part by weight to about 10 parts by weight, about 0.2 part by weight to about 5 parts by weight, about 0.3 part by weight to about 2 parts by weight, based on 100 parts by weight of the simulated vernix composition.

In some embodiments, the vernix lipid includes a ceramide. In some embodiments, the vernix lipid consists of a ceramide. Non-limiting examples of ceramides include ceramide II, ceramide III (also called ceramide NP), ceramide analogs (such as vegetable derived ceramides), and the like. In some embodiments, an amount of the ceramides ranges from about 0.05 parts by weight to about 10 parts by weight, such as about 0.1 part by weight to about 5 parts by weight, about 0.2 part by weight to about 2.5 parts by weight, about 0.3 part by weight to about 1 part by weight, based on 100 parts by weight of the simulated vernix composition.

In some embodiments, the vernix lipid includes a cholesterol or a plant based sterol (phytosterol), such as a plant based beta sitosterol. In some embodiments, an amount of the cholesterol or plant based sterol ranges from about 0.1 part by weight to about 80 parts by weight, such as about 0.2 part by weight to about 50 parts by weight, about 0.3 part by weight to about 40 parts by weight, about 0.5 part by weight to about 20 parts by weight, about 1 part by weight to about 15 parts by weight, about 2 parts by weight to about 10 parts by weight, about 5 parts by weight to about 7.5 parts by weight, based on 100 parts by weight of the simulated vernix composition. In some embodiments, the an amount of the cholesterol is about 0.1 part by weight, about 0.2 part by weight, about 0.5 part by weight, about 1 part by weight, about 2 parts by weight, about 5 parts by weight, about 10 parts by weight, about 20 parts by weight, about 30 parts by weight, about 40 parts by weight, about 50 parts by weight, about 60 parts by weight, about 70 parts by weight, about 80 parts by weight, or any ranges therebetween.

In some embodiments, the vernix lipid includes a phospholipid. Non-limiting examples of phospholipids include glycerophospholipids and sphingophospholipids. Non-limiting examples of glycerophospholipids include phosphatidic acid (phosphatidate) (PA), phosphatidylethanolamine (cephalin) (PE), phosphatidylcholine (lecithin) (PC), phosphatidylserine (PS), phosphoinositides: phosphatidylinositol (PD), phosphatidylinositol phosphate (PIP), phosphatidylinositol bisphosphate (PIP2), and the like. Non-limiting examples of sphingophospholipids include ceramide phosphorylcholine (Sphingomyelin) (SPH), ceramide phosphorylethanolamine (Sphingomyelin) (Cer-PE), ceramide phosphoryllipid, cerebrosides, gangliosides, and the like. In some embodiments, the phospholipid includes lecithin. In some embodiments, the vernix lipid includes lecithin, and the lecithin is obtained from egg yolk, soybeans, or the like. In some embodiments, an amount of the phospholipid ranges from about 0.5 part by weight to about 15 parts by weight, such as about 0.75 part by weight to about 10 parts by weight, about 0.5 part by weight to about 7.5 parts by weight, about 1 part by weight to about 5 parts by weight, based on 100 parts by weight of the simulated vernix composition.

In some embodiments, the vernix lipid includes a wax diester. Non-limiting examples of wax diesters include carnauba wax, bees wax, candelilla wax, or the like. In certain embodiments, wax diesters can include at least one of propyl laurate, lauryl laurate, myristyl laurate, palmityl laurate, stearyl laurate, arachidyl laurate, behenyl laurate, myristoleyl laurate, palmitoleyl laurate, oleyl laurate, linoleyl laurate, linolenyl laurate, lauryl myristate, myristyl myristate, palmityl myristate, stearyl myristate, arachidyl myristate, behenyl myristate, myristoleyl myristate, palmitoleyl myristate, oleyl myristate, linoleyl myristate, linolenyl myristate, 1,2-cicosandiol-dipentadecanoate, propyl palmitate, lauryl palmitate, myristyl palmitate, palmityl palmitate, stearyl palmitate, arachidyl palmitate, behenyl palmitate, myristoleyl palmitate, palmitoleyl palmitate, oleyl palmitate, linoleyl palmitate, linolenyl palmitate, heptadecanyl heptadecanoate, isopropyl stearate, propyl stearate, lauryl stearate, myristyl stearate, palmityl stearate, heptadecanoyl stearate, stearyl stearate, arachidyl stearate, behenyl stearate, myristoleyl stearate, palmitoleyl stearate, oleyl stearate, and/or linoleyl stearate. In some embodiments, an amount of the wax diester ranges from about 0.5 part by weight to about 20 parts by weight, such as about 1 part by weight to about 15 parts by weight, about 2 parts by weight to about 10 parts by weight, about 3 parts by weight to about 7.5 part by weight, based on 100 parts by weight of the simulated vernix composition.

In some embodiments, the vernix lipid includes a glycosphingolipid. Non-limiting examples of glycosphingolipids includes those commonly referred to as “vegetable ceramide,” those sold under the trade name CERAMOSIDES™ HP by Seppic, and the like. Ceramides useful in certain embodiments can be prepared synthetically or by fermentation. In certain embodiments, glycosphingolipids can include at least one of FSS vegetable Ceramides BG (Formulator Sample Shop), AC Vegetable Ceramides G (Active Concepts), Ceramide Vegetable Extract (Soap & More), Vegetable Ceramide (Skin Foodie), Vegetable Ceramides (CosmoCan), and/or Vegetable Ceramides bgg biofunctional (Ashland). In some embodiments, an amount of the glycosphingolipid ranges from about 0.05 parts by weight to about 10 parts by weight, such as about 0.1 part by weight to about 5 parts by weight, about 0.2 part by weight to about 2.5 parts by weight, about 0.3 part by weight to about 1 part by weight, based on 100 parts by weight of the simulated vernix composition.

In some embodiments, the simulated vernix composition includes a cholesterol derivative, such as cholesterol sulfate, or beta sitosterol. In some embodiments, an amount of the cholesterol derivative ranges from about 0.01 part by weight to about 1 part by weight, such as about 0.02 part by weight to about 0.5 parts by weight, about 0.05 parts by weight to about 0.3 part by weight, about 0.1 part by weight to about 0.5 part by weight, based on 100 parts by weight of the simulated vernix composition.

In some embodiments, the simulated vernix composition includes a fatty acid. In some embodiments, the fatty acid can include, for example, lauric acid, myristic acid, palmitic acid, stearic acid, arachidic acid, behenic acid, arachidonic acid, linoleic acid, alpha-linolenic acid, oleic acid, or other fatty acid, or mixtures thereof.

In some embodiments, the simulated vernix composition includes a fatty alcohol. In some embodiments, the fatty acid can include, for example, lauryl alcohol, myristyl alcohol, cetyl alcohol, stearyl alcohol, oleyl alcohol, arachidyl alcohol, behenyl alcohol, or other fatty alcohol, or mixtures thereof.

In some embodiments, the simulated vernix composition includes a lanolin alcohol.

In some embodiments, the vernix composition is an animal free or vegan vernix composition, which lacks lanolin alcohol and/or cholesterol. In some embodiments, the animal free/vegan vernix composition beta sitosterol and/or glyceryl behenate.

In some embodiments, the vernix composition includes tocopherol. In some embodiments, the non-animal free/non-vegan vernix composition includes tocopherol. In some embodiments, the animal free/vegan vernix composition includes tocopherol. In some embodiments, the tocopherol prevents oxidation of unsaturated fatty acids present in the composition.

In some embodiments, instead of the vernix lipid, the simulated vernix composition includes a lipid that is different from the lipids found in vernix or similar lipids.

In some embodiments, the simulated vernix composition includes: 1 part by weight to 5 parts by weight of the cholesterol ester, 0.5 part by weight to 3 parts by weight of the cholesterol, 0.5 part by weight to 3 parts by weight of the phospholipid, 0.5 part by weight to 3 parts by weight of the wax diester, 0.5 part by weight to 3 parts by weight of the glycosphingolipid, 0.1 part by weight to 1 part by weight of a cholesterol sulfate, 0.1 part by weight to 1 part by weight of the ceramide, or combinations thereof, based on 100 parts by weight of the simulated vernix composition. In some embodiments, the simulated vernix composition includes: 1 part by weight to 5 parts by weight of the cholesterol ester, 0.5 part by weight to 3 parts by weight of the cholesterol, 0.5 part by weight to 3 parts by weight of the phospholipid, 0.5 part by weight to 3 parts by weight of the wax diester, 0.5 part by weight to 3 parts by weight of the glycosphingolipid, 0.1 part by weight to 1 part by weight of a cholesterol sulfate, and 0.1 part by weight to 1 part by weight of the ceramide, based on 100 parts by weight of the simulated vernix composition. According to certain embodiments, the amount of the vernix lipids does not exceed 20 parts by weight based on 100 parts by weight of the simulated vernix composition.

In some embodiments, the simulated vernix composition is an oil phase mixture. In some embodiments, the simulated vernix composition is a uniform oil phase mixture. In some embodiments, the simulated vernix composition is an oil phase solution. The present study discovered, in one aspect, that squalane is a poor solvent for vernix lipids such as ceramides. When the amount of squalane is about 50 parts by weight or more based on 100 parts by weight of the simulated vernix composition, the vernix lipid does not fully dissolve and a turbid mixture is obtained. The inclusion of the liquid monoester as well as the method of preparing the simulated vernix composition as described below are able to overcome the solubility issue (especially at elevated temperatures of 40° C. or more, such as 50° C. or more, 60° C. or more, 70° C. or more, 80° C. or more, 90° C. or more, 100° C. or more, or 120° C. or more) and result in the uniform oil phase mixture or the oil phase solution. The ability of the simulated vernix composition herein to become a clear solution allows the formation of a uniform semi-solid composition at room temperature (about 20° C. or about 25° C.).

### Method of Preparing Simulated Vernix Composition

In some embodiments, the present invention is directed to a method of preparing a simulated vernix composition. In some embodiments, the simulated vernix composition is the same as or similar to those as described elsewhere herein, such as the “Simulated Vernix Composition” section.

In some embodiments, the method includes heating squalane to an elevated temperature; mixing components for the simulated vernix composition other than squalane with the squalane while maintaining the mixture at the elevated temperature; and cooling down the mixture.

In one aspect, the present study discovered that, without heating the squalane and the mixture during the mixing, some or all of the vernix lipid(s) would not dissolve in the squalane and a hazy or turbid mixture would be resulted even if the mixture includes the liquid monoester (the liquid monoester is described elsewhere herein, such as in the “Simulated Vernix Composition” section). Without adding the liquid monoester to the mixture, even though the vernix lipid(s) sometimes dissolves in the squalane at the elevated temperature, precipitate forms in the mixture when the temperature drops, and a hazy or turbid mixture would be resulted, as well.

In some embodiments, the components for the simulated vernix composition are mixed at the elevated temperature until the mixture becomes clear before the mixture is cooled down.

In some embodiments, the elevated temperature is about 75° C. or higher, such as about 80° C. or higher, about 85° C. or higher, or about 90° C. or higher.

In some embodiments, the mixture is stirred during the mixing step at the elevated temperature.

### Emulsion Including Simulated Vernix Composition

In some aspects, the present invention is directed to an emulsion including an oil phase simulated vernix composition; and water. In some embodiments, the oil phase simulated vernix composition is the same as or similar to those as described elsewhere herein, such as in the “Simulated Vernix Composition” section.

In some embodiments, the emulsion is an oil-in-water emulsion. In some embodiments, the emulsion is a water-in-oil emulsion.

In some embodiments, the emulsion further includes a surfactant/an emulsifier. In the instant specification, the terms “surfactant” and “emulsifier” are considered as the same class of ingredients and are used interchangeably.

In some embodiments, an amount of the surfactant ranges from about 0.05 parts by weight to about 18 parts by weight, such as about 0.1 part by weight to about 15 parts by weight, about 0.2 part by weight to about 10 parts by weight, about 0.5 part by weight to about 7.5 parts by weight, based on 100 parts by weight of the emulsion.

In some embodiments, the surfactant includes a nonionic surfactant.

Non-limiting examples of nonionic surfactants include monomeric, homopolymeric and block copolymeric ethers. In some embodiments, such ethers are formed by the polymerization of monomeric alkylene oxides, such as ethylene oxide or propylene oxide.

Further non-limiting examples of nonionic surfactants include alkoxylated sorbitan and alkoxylated sorbitan derivatives. For example, alkoxylation, such as ethoxylation, of sorbitan provides polyalkoxylated sorbitan derivatives. Esterification of polyalkoxylated sorbitan provides sorbitan esters such as the polysorbates. Non-limiting examples of such ingredients include Polysorbates 20-85, sorbitan oleate, sorbitan palmitate, sorbitan sesquiisostearate, sorbitan stearate, and so on.

Further non-limiting examples of nonionic surfactants include silicone surfactants, which are defined as silicone polymers, having at least one hydrophilic radical and at least one lipophilic radical. In some embodiments, the silicone surfactants include organosiloxane polymers that are liquids or solids at room temperature. In some embodiments, the organosiloxane surfactant is a water-in-oil or oil-in-water type surfactants. In some embodiments, the organosiloxane surfactant has a Hydrophile/Lipophile Balance (HLB) of ranging from about 2 to about 18, such as about 2 to about 12, about 2 to about 10, or about 4 to about 6. In some embodiments, the HLB of a nonionic surfactant is the balance between the hydrophilic and lipophilic portions of the surfactant and is calculated according to the following formula:

$$HLB = 7 + 11.7 \times log Mw / Mo ,$$

where Mw is the molecular weight of the hydrophilic group portion and Mo is the molecular weight of the lipophilic group portion.

Non-limiting examples of silicone surfactants include those sold by Dow Corning under the tradename Dow Corning 3225C Formulation Aid, Dow Corning 190 Surfactant, Dow Corning 193 Surfactant, Dow Corning Q2-5200, and the like. Further non-limiting examples of silicone surfactants include surfactants sold under the tradename SILWET by Union Carbide, and surfactants sold by Troy Corporation under the TROYSOL tradename, those sold by Taiwan Surfactant Co. under the tradename ABLUSOFT, those sold by Hoechst under the tradename ARKOPHOB.

In some embodiments, the nonionic surfactant includes an alkoxylated alcohol or ether formed by the reaction of a fatty acid with a polyhydric alcohol such as glucose or methyl glucose and an ethoxylated alcohol; a sorbitan derivative, and mixtures thereof. In some embodiments, the nonionic surfactant includes a nonionic surfactant selected from polyoxyethylene 3-oleyl ether, Polysorbate 20, sorbitan stearate, PEG-20 methyl glucose sesquiisostearate, and combinations thereof.

In some embodiments, the surfactant includes a cationic surfactant, an anionic surfactant, a zwitterionic surfactant, and an amphoteric surfactant. Examples of cationic, anionic, zwitterionic, and amphoteric surfactants are disclosed in U.S. Pat. No. 5,534,265, which is hereby incorporated by reference in its entirety.

### Additional Components

In some embodiments, the simulated vernix composition and/or the emulsion as described herein further includes one or more additional components.

In some embodiments, the simulated vernix composition and/or the emulsion as described herein is an ingredient of a cosmetic composition, a skin care composition, a personal care composition, and/or a pharmaceutical composition which includes, apart from the simulated vernix composition and/or the emulsion, one or more additional components. The skin care composition is also described elsewhere herein, such as in the “Skin Care Composition Including Simulated Vernix Composition or Emulsion Thereof” section.

Non-limiting examples of the additional components include the following:

### Particulate Matter:

For a color cosmetic, the simulated vernix composition, the emulsion, the cosmetic composition, the skin care composition, the personal care composition, and/or the pharmaceutical composition sometimes include 1-20%, such as 1.5-18% of particulate matter, based on 100% of the weight of the simulated vernix composition, the emulsion, the cosmetic composition, the skin care composition, the personal care composition, and/or the pharmaceutical composition. In some embodiments, the particulate matter has a particle size ranging from about 0.01 micron to about 200 microns, such as 0.25 micron to 100 microns. In some embodiments, the particulate matter is colored or non-colored (for example white) non-pigmentitious powders sometimes give the cosmetic stick an opaque or semi-opaque quality and contribute to stick structure. Non-limiting examples of non-pigmentatious powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. While titanium dioxide is commonly considered to be a white pigment when used in paints, in cosmetic sticks it is used more for its ability to mute color, and/or provide an opaque or semi-opaque finish, then as a colorizing ingredient. The above-mentioned powders are sometimes surface treated with lecithin, amino acids, mineral oil, silicone, or various other agents either alone or in combination, which coat the powder surface and render the particles more lipophilic in nature.

In some embodiments, the particulate matter component includes various organic and/or inorganic pigments, alone or in admixture with one or more non-pigmentatious powders. The organic pigments are sometimes various aromatic types including azo, indigoid, triphenylmethane anthraquinone, and xanthine dyes which are designated as D&C and FD&C blues, browns, greens, oranges, reds, yellows, and so forth. Organic pigments generally comprise insoluble metallic salts of certified color additives, referred to as the Lakes. Inorganic pigments include iron oxides, ultramarines, chromium, chromium hydroxide colors, and mixtures thereof.

In some embodiments, the simulated vernix composition, the emulsion, the cosmetic composition, the skin care composition, the personal care composition, and/or the pharmaceutical composition includes a mixture of both pigmentatious and non-pigmentatious particulate matter. The percentage of pigment used in the particulate matter component will depend on the type of cosmetic being formulated.

### Humectant:

In some embodiments, the simulated vernix composition, the emulsion, the cosmetic composition, the skin care composition, the personal care composition, and/or the pharmaceutical composition is a makeup or color cosmetic composition. In some embodiments, the makeup or color cosmetic composition includes a humectant.

In some embodiments, an amount of the humectant is about 0.1-30%, such as about 0.5-25%, about 1-20% based on the total weight of the makeup or color cosmetic composition.

Suitable humectants include materials glycols, sugars, and similar materials. Suitable glycols include polyethylene and polypropylene glycols such as PEG 4-240, which are polyethylene glycols having from 4 to 240 repeating ethylene oxide units; as well as C1-6 alkylene glycols such as propylene glycol, butylene glycol, and the like. Suitable sugars, some of which are also polyhydric alcohols, are also suitable humectants. Examples of such sugars include glucose, fructose, honey, hydrogenated honey, inositol, maltose, mannitol, maltitol, sorbitol, sucrose, xylitol, xylose, and so on. Preferably, the humectants used in the composition of the invention are C1-6, preferably C2-4 alkylene glycols, most particularly butylene glycol.

### Gellants:

In some embodiments, the simulated vernix composition, the emulsion, the cosmetic composition, the skin care composition, the personal care composition, and/or the pharmaceutical composition further includes a gellant.

Gellants in the oil or water phase are sometimes able to provide desirable viscosity or gelling properties, and is sometimes able to formulate the composition or the emulsion in the form of a solid stick.

In some embodiments, an amount of the gellant in the simulated vernix composition, the emulsion, the cosmetic composition, the skin care composition, the personal care composition, and/or the pharmaceutical composition is about 1-20%, such as 3-18% or 5-10% based on the total weight of the simulated vernix composition, the emulsion, the cosmetic composition, the skin care composition, the personal care composition, and/or the pharmaceutical composition.

### Oils:

In some embodiments, the simulated vernix composition, the emulsion, the cosmetic composition, the skin care composition, the personal care composition, and/or the pharmaceutical composition further includes an oil.

In some embodiments, an amount of the oil is about 0.1-50%, such as about 0.5-40%, or about 1-35% based on the total weight of the simulated vernix composition, the emulsion, the cosmetic composition, the skin care composition, the personal care composition, and/or the pharmaceutical composition.

The oils used may be volatile or nonvolatile. The term “volatile” means that the oil has a measurable vapor pressure, or a vapor pressure of at least 2 mm. of mercury at 20° C. The term “nonvolatile” means that the oil has a vapor pressure of less than 2 mm. of mercury at 20° C. If the cosmetic compositions are transfer resistant sticks, it is desirable to use significant amounts of volatile solvent for the oil component. Suitable volatile solvents or oils are liquids, and enable easy formulation of the cosmetic stick of the invention. When the cosmetic stick product is applied to skin or lips, the volatile solvent of the invention must be capable of flashing off to leave the other ingredients in the stick on the skin. Suitable volatile solvents sometimes have a viscosity of 0.5 to 10 centistokes at 25° C. Suitable volatile oils include linear silicones, cyclic silicones, paraffinic hydrocarbons, or mixtures thereof.

Also suitable as the volatile oils are various straight or branched chain paraffinic hydrocarbons having 5 to 40 carbon atoms, more preferably 8-20 carbon atoms. Suitable hydrocarbons include pentane, hexane, heptane, decane, dodecane, tetradecane, tridecane, and C8-20 isoparaffins as disclosed in U.S. Pat. Nos. 3,439,088 and 3,818,105, both of which are hereby incorporated by reference. Exemplary volatile paraffinic hydrocarbons have a molecular weight of 70-225, such as 160 to 190 and a boiling point range of 30 to 320° C., such as 60-260° C., and a viscosity of less than 10 cs. at 25° C. Such paraffinic hydrocarbons are available from EXXON under the ISOPARS trademark, and from the Permethyl Corporation. Suitable C12 isoparaffins are manufactured by Permethyl Corporation under the tradename Permethyl 99A. Another C12 isoparaffin (isododecane) is distributed by Presperse under the tradename PERMETHYL 99A. Various C16 isoparaffins commercially available, such as isohexadecane (having the tradename Permethyl R), are also suitable. Transfer resistant cosmetic sticks of the invention will generally comprise a mixture of volatile silicones and volatile paraffinic hydrocarbons.

A wide variety of nonvolatile oils are also suitable for use in the instant cosmetic composition. The nonvolatile oils generally have a viscosity of greater than 10 centipoise at 25° C., and sometimes range in viscosity up to 1,000,000 centipoise at 25° C. Examples of nonvolatile oils suitable for use in the cosmetic sticks include esters of the formula RCO—OR^{A} wherein R and RA are each independently a C1-25, preferably a C4-20 straight or branched chain alkyl, alkenyl or alkoxycarbonylalkyl or alkylcarbonyloxyalkyl. Examples of such esters include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, cocodicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, and fatty alcohols such as oleyl alcohol, isocetyl alcohol, and the like, as well as the esters disclosed on pages 24-26 of the C.T.F.A. Cosmetic Ingredient Handbook, First Edition, 1988, which is hereby incorporated by reference.

The oil may also comprise naturally occurring glyceryl esters of fatty acids, or triglycerides. Both vegetable and animal sources may be used. Examples of such oils include castor oil, lanolin oil, triisocetyl citrate, C10-18 triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

Also suitable as the oil are synthetic or semi-synthetic glyceryl esters, e.g., fatty acid mono-, di-, and triglycerides which are natural fats or oils that have been modified, for example, acetylated castor oil, glyceryl stearate, glyceryl dioleale, glyceryl distearate, glyceryl trioctanoate, glyceryl distearate, glyceryl linoleate, glyceryl myristate, glyceryl isostearate, PEG castor oils, PEG glyceryl oleates, PEG glyceryl stearates, PEG glyceryl tallowates, and so on.

Also suitable as the oil are nonvolatile hydrocarbons such as isoparaffins, hydrogenated polyisobutene, mineral oil, squalene, petrolatum, and so on.

Straight or branched chain fatty alcohols having the formula R—OH, wherein R is a straight or branched chain saturated or unsaturated alkyl having 6-30 carbon atoms, are also suitable oils. Such fatty alcohols include cetyl alcohol, stearyl alcohol, cetearyl alcohol, and the like.

Also suitable as the oil are various lanolin derivatives such as acetylated lanolin, acetylated lanolin alcohol, and so on.

Nonvolatile silicones, both water soluble and water insoluble, are also suitable as the oil component. Such silicones preferably have a viscosity of 10 to 600,000 centistokes, preferably 20 to 100,000 centistokes at 25° C. Suitable water insoluble silicones include amodimethicone, bisphenylhexametlticone, dimethicone, hexadecyl methicone, methicone, phenyl trimethicone, simethicone, dimethylhydrogensiloxane, stearoxytrimethylsilane, vinyldimethicone, and mixtures thereof. Also suitable are water soluble silicones such as dimethicone copolyol, dimethiconol, and the like. Such silicones are available from Dow Corning as the 3225C formulation aid, Dow 190 and 193 fluids, or similar products marketed by Goldschmidt under the ABIL tradename.

Also suitable as the nonvolatile oil are various fluorinated oils such as fluorinated silicones, fluorinated esters, or perfluropolyethers. Particularly suitable are fluorosilicones such as trimethylsilyl endcapped fluorosilicone oil, polytrifluoropropylmethylsiloxanes, and similar silicones such as those disclosed in U.S. Pat. No. 5,118,496 which is hereby incorporated by reference. Perfluoropolyethers like those disclosed in U.S. Pat. Nos. 5,183,589, 4,803,067, and 5,183,588, all of which are hereby incorporated by reference, which are commercially available from Montefluos under the trademark Fomblin, are also suitable shine enhancers.

Guerbet esters are also suitable oils. The term “guerbet ester” means an ester which is formed by the reaction of a guerbet alcohol. Preferably, the guerbet ester is a fluoro-guerbet ester which is formed by the reaction of a guerbet alcohol and carboxylic acid (as defined above). Examples of suitable fluoro guerbet esters are set forth in U.S. Pat. No. 5,488,121 which is hereby incorporated by reference. Suitable fluoro-guerbet esters are also set forth in U.S. Pat. No. 5,312,968 which is hereby incorporated by reference. Exemplary is a guerbet ester having the tentative CTFA name fluoro-octyldodecyl meadowfoamate. This ester is sold by Siltech, Norcross Georgia as Developmental Ester L61125A, under the tradename Silube GMEF.

In some embodiments, the compositions of the invention further include a water insoluble nonvolatile silicone having a viscosity of about 5-25 centipoise at 25° C., such as dimethicone. In some embodiments, the silicone serves as a formulation aid.

### Preservatives:

In some embodiments, the simulated vernix composition, the emulsion, the cosmetic composition, the skin care composition, the personal care composition, and/or the pharmaceutical composition includes a preservative.

In some embodiments, an amount of the preservative is about 0.001-8%, such as 0.01-6%, or 0.05-5% based on a total weight of the simulated vernix composition, the emulsion, the cosmetic composition, the skin care composition, the personal care composition, and/or the pharmaceutical composition.

A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, phenoxyethanol, methyl paraben, propyl paraben, diazolidinyl urea, calcium benzoate, calcium propionate, captan, chlorhexidine diacetate, chlorhexidine digluconate, chlorhexidine dihydrochloride, chloroacetarnide, chlorobutanol, p-chloro-m-cresol, chlorophene, chlorothymol, chloroxylenol, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

### Skin Care Composition Including Simulated Vernix Composition or Emulsion Thereof

In some embodiments, the simulated vernix composition, the emulsion thereof is a component of a skin care composition. Examples of skin care compositions include a lotion, a cream, a gel, a sunscreen composition, an ointments, a solid, a mull, and the like.

Suitable skin care lotions and creams include those in the emulsion form, and may be water-in-oil or oil-in-water emulsions, such as oil-in-water emulsions. In some embodiments, the creams, lotions, gels and/or sunscreens may contain the following ranges of ingredients:
0.01-50% simulated vernix composition,
0.5-95% water,
0.1-50% oil, and
0.01-20% surfactant.

The suitable oil and surfactant are described elsewhere herein.

### Skin Treating Active Ingredients:

In some embodiments, the skin care composition includes one or more skin-treating active ingredients.

In some embodiments, an amount of the one or more skin-treating active ingredients is about 0.1-15%, such as 0.5-10% or 1-8% based on the total weight of the skin care composition. Non-limiting examples of skin treating active agents include peptides, retinols, niacinamide, botanical extracts, acidic keratolytic or anti-acne agents, and the like.

### Sunscreens:

In some embodiments, the skin care composition is the sunscreen composition. In some embodiments, the skin care composition includes one or more sunscreens.

In some embodiments, an amount of the one or more sunscreens is about 0.001-20%, such as 0.01-10% or 0.05-8% based on a total weight of the skin care composition.

A sunscreen is defined as an ingredient that absorbs at least 85 percent of the light in the UV range at wavelengths from 290 to 320 nanometers, but transmit UV light at wavelengths longer than 320 nanometers. Sunscreens generally work in one of two ways. Particulate materials, such as zinc oxide or titanium dioxide, physically block ultraviolet radiation. Chemical sunscreens, on the other hand, operate by chemically reacting upon exposure to UV radiation. Suitable sunscreens that may be included in the compositions of the invention are set forth on page 582 of the CTFA Cosmetic Ingredient Handbook, Second Edition, 1992, as well as U.S. Pat. No. 5,620,965, both of which are hereby incorporated by reference. Examples of such sunscreen materials are p-aminobenzoic acid (PABA), cinoxate, diethanolamine p-methoxycinnamate (DEA-methoxycinnamate), Digalloyl trioleate, dioxybenzone (Benzophenone-8), ethyl 4-[bis-(hydroxypropyl)]amnobenzoate (ethyl dihydroxypropyl PABA), 2-ethylhexyl-2-cyano-3,3-diphenylacrylate (octocrylene), ethylhexyl p-methoxycinnamate (Octyl methoxycinnamate), 2-ethylhexyl salicylate (Octyl salicylate), glyceryl aminobenzoate (Glyceryl PABA), homosalate, lawsone with dihydroxyacetone, menthyl anthranilate, oxybenzone (Benzophenone-3), Padimate A (Pentyl Dimethyl PABA), Padimate O, (Octyl Dimethyl PABA), 2-Phenylbenzimidazole-5-sulfonic acid (Phenylbenzimidazole Sulfonic acid), Red Petrolatum, Sulisobenzone (Benzophenone-4), triethanolamine salicylate (TEA-Salicylates), and so on.

### Vitamins and Antioxidants:

In some embodiments, the skin care compositions further include a vitamin and/or a coenzyme, or an antioxidant.

In some embodiments, an amount of the vitamins and/or coenzymes is about 0.001-10%, such as 0.01-8% or 0.05-5% based on the total weight of the skin care composition.

Suitable vitamins include ascorbic acid and derivatives thereof, the B vitamins such as thiamine, riboflavin, pyridoxin, and so on, as well as coenzymes such as thiamine pyrophosphate, flavin adenine dinucleotide, folic acid, pyridoxal phosphate, tetrahydrofolic acid, and so on. Also, Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D and K are suitable.

Suitable antioxidants are ingredients which assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

### EXAMPLES

The instant specification further describes in detail by reference to the following experimental examples. These examples are provided for purposes of illustration only, and are not intended to be limiting unless so specified. Thus, the instant specification should in no way be construed as being limited to the following examples, but rather, should be construed to encompass any and all variations which become evident as a result of the teaching provided herein.

### Example 1, 2 and 3: Simulated Vernix Composition

Example 1 and 2 are non-limiting examples of simulated vernix composition in accordance with some embodiments. Example 3 is a similar composition, but does not include any liquid esters as described herein. The compositions of Examples 1-3 are listed below in Table 2:

TABLE 2
Compositions of Examples 1-3 (in Weight Percentage)

| Formula # | Example 1 | Example 2 | Example 3 |
|---|---|---|---|
| Squalane | 70.5 | 70.5 | 90.5 |
| Polyglyceryl-4 Isostearate | 20 | 0 | 0 |
| Jojoba Oil | 0 | 20 | 0 |
| Lecithin | 3 | 3 | 3 |
| Cholesterol | 6 | 6 | 6 |
| Ceramide II | 0.5 | 0.5 | 0.5 |

To make the composition described in Example 1, 2, and 3, squalane was heated to 95° C., and then the remaining ingredients were added to squalane one by one at 95° C.

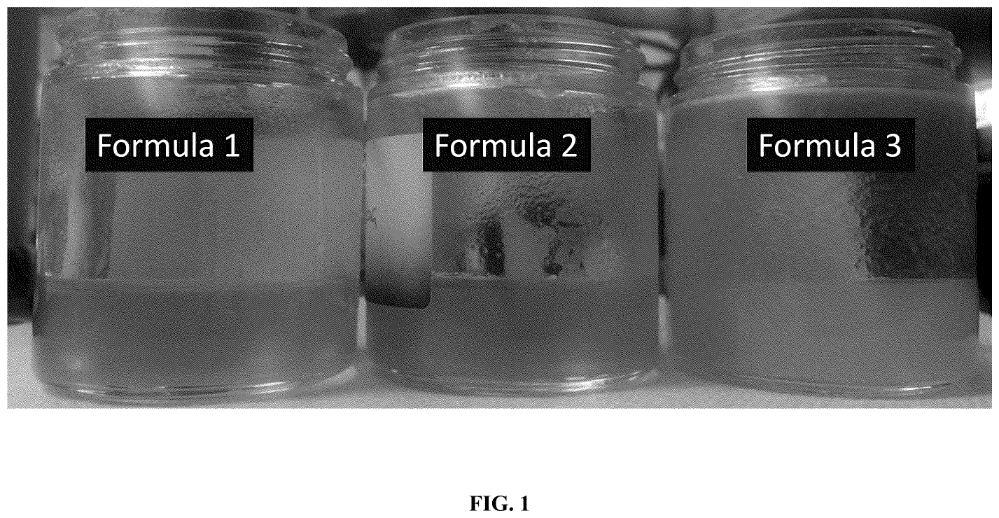

Ceramide II does not solubilize in oils, such as squalane. As shown in FIG. 1, the formulations of Examples 1 and 2 are clear solutions, while ceramide II precipitated out of the mixture of Example 3. As a result, clear and uniform oil phase solutions were obtained for Examples 1 and 2, while Example 3 was a white and hazy solution, indicating the lipids in Example 3 are not compatible with squalane.

### Example 4 and 5: Simulated Vernix Composition Versus Similar Composition without Vernix Lipids

Example 4 and 5 were used to show the effectiveness of simulated vernix by consumer self-evaluation test.

The composition of Example 4 included several vernix lipids and was a simulated vernix composition. The composition of Example 5 is similar to Example 4, but does not include any vernix lipids. The compositions of Examples 4 and 5 are shown below in Table 3:

TABLE 3
Compositions of Examples 4 and 5 (in Weight Percentage)

| Formula# | Example 4 | Example 5 |
|---|---|---|
| Squalane | 63.8 | 80 |
| Jojoba Oil | 20 | 20 |
| Carnauba wax | 5 | 0 |
| Lecithin | 3 | 0 |
| Cholesterol | 6 | 0 |
| Ceramide 2 | 0.2 | 0 |
| Ceramide 3 | 0.3 | 0 |
| C10-30 Cholesterol/Lanosterol Esters | 3 | 0 |
| Glycosphingolipids and Glycolipids | 2.4 | 0 |

To make the composition described in Example 4 and 5, the squalane was heated to 95° C., and then remaining ingredients were added into squalane one by one while maintaining the mixture at 95° C. The efficacies of the compositions Examples 4 & 5 were tested as follows:

### Test Procedure

The consumer evaluation for the composition of Example 4 & 5 was done by an expert panel. In the testing procedure, the present study identified 20 people with sensitive skin and have experienced either skin itchiness or/and skin irritation. The double-blind consumer panel study was carried out as follows. The composition of Example 4 or 5 was applied in a split face testing design, in the morning and night for 4 weeks. The split face testing in the present invention, Example 4 or 5 was applied to half of the face according to a randomization table. Over the course of the next 4 weeks, the consumer answered a questionnaire about skin itchiness and/or skin irritation. Two consumers withdrew during the 4 weeks of testing. Consumer rankings of the effectiveness of treatment are summarized below in Table 4:

TABLE 4
Consumer Evaluation Results (graded
according to the number of persons)

| Formula # | Example 4 | Example 5 |
|---|---|---|
| Very Effective | 4 Persons | 1 Person |
| Effective | 9 Persons | 5 Persons |
| Somewhat Effective | 3 Persons | 6 Persons |
| Not Effective | 2 Persons | 6 Persons |

Table 4 shows the consumer rankings of the effectiveness of treatment by the compositions of Example 4 or 5. The evaluation results show that there is a significant improvement with the simulated vernix to reduce the symptoms of skin itchiness and irritation after 4 weeks of use. As such, the composition of Example 4 will be used as the simulated vernix composition in the following Examples.

### Example 6: Serum for Sensitive Skin

A non-limiting example of skin care composition (a serum for treating sensitive skin) including the simulated vernix composition of Example 4 was prepared. The ingredients are listed below in Table 5:

TABLE 5
Serum for Sensitive Skin Ingredients

| Ingredient | Weight Percentage |
|---|---|
| Squalane | 97.10 |
| Simulated Vernix (Example 4) | 2.00 |
| Evening Prime Rose | 0.50 |
| Bisabolol | 0.10 |
| Pelargonium Graveolens Oil | 0.10 |
| Hydrasalinol | 0.20 |

To make the composition described in Example 6, squalane was heated to 80° C., and then the remaining ingredient were added one by one while maintaining the temperature of the mixture at 80° C.

### Example 7: Anti-Aging Cream

A non-limiting example of skin care composition (an anti-aging cream) including the simulated vernix composition of Example 4 was prepared. The ingredients are listed below in Table 6:

TABLE 6
Anti-Aging Cream Ingredients

| Seq | Ingredient | Weight Percentage |
|---|---|---|
| 1 | DI Water | 60.86 |
| 1 | Disodium EDTA | 0.10 |
| 1 | 1,3-Propanediol | 5.00 |
| 1 | Sodium Hyaluronate | 0.05 |
| 1 | Carbopol 980 | 0.04 |
| 1 | Aloe Vera Juice | 5.00 |
| 2 | Glycerin | 4.00 |
| 2 | Xanthan Gum | 0.20 |
| 3 | Simulated Vernix of Example 4 | 3.00 |
| 3 | Dicaprylyl Carbonate | 3.00 |
| 3 | Montanov 202 | 3.00 |
| 3 | Cetearyl Alcohol | 0.75 |
| 3 | Polysorbate 60 | 0.30 |
| 3 | Vitamin E Acetate | 0.20 |
| 3 | Sweet Almond Oil | 2.00 |
| 3 | Cetyl Alcohol | 0.50 |
| 3 | Dimethicone | 0.90 |
| 3 | Bisabolol | 1.00 |
| 3 | Murumuru Butter | 0.20 |
| 4 | DI Water | 4.00 |
| 5 | Peptide Solution | 5.00 |
| 6 | Euxyl PE 9010 | 0.90 |

To make the composition described in Example 7, the ingredients of Seq. 1 were added to the main kettle under propeller agitation (350-450 RPM) until uniform. In an auxiliary kettle, Seq. 2 ingredients were added and mix under propeller agitation until uniform. The premixed Seq. 2 were then added to the main kettle including mixed Seq. 1 ingredients and mix for 10-15 minutes. In an auxiliary kettle, the ingredients of Seq. 3 were added, heated to 78° C.-80° C. and subjected to propeller agitation until uniform. The mixtures of Seq. 3 ingredients were added to the main kettle and subjected to homogenizer agitation (3000-4000 RPM) for 10-15 minutes, and then to propeller agitation and cooled down to 30° C. In an auxiliary kettle, the materials of Seq. 4-6 were mix under propeller agitation until uniform. The premixed Seq. 4-6 were then added to the main kettle and mix until uniform. The pH of the final batch was adjusted to 6.5.

### Example 8 Moisturizing Long Wear Foundation Composition

A non-limiting example of skin care composition (a composition for moisturizing long wear foundation) including the simulated vernix composition of Example 4 was prepared. The skin care composition of Example 8 was formulated to address the skin dryness, which is a major drawback of long wear foundation. The ingredients are listed below in Table 7:

TABLE 7
Moisturizing Long Wear Foundation Ingredients

| Seq | Ingredient | Weight Percentage |
|---|---|---|
| 1 | DI Water | 40.13 |
| 1 | Laureth-7 | 1.00 |
| 1 | Barguard CP | 0.70 |
| 1 | Magnesium Sulfate Heptahydrate | 0.90 |
| 2 | Butylene Glycol | 3.00 |
| 3 | Dow Corning 5225C | 6.50 |
| 3 | Plurol Diissostearaquie CG | 0.45 |
| 3 | Cyclopentasiloxane | 4.00 |
| 3 | Mica/Methicone | 3.00 |
| 3 | Polymethylsilsesquioxane | 0.50 |
| 3 | Hydrophobic Micro TiO₂ | 3.00 |
| 3 | Hydrophobic Yellow Iron Oxide | 3.40 |
| 3 | Hydrophobic Red Iron Oxide | 0.95 |
| 3 | Hydrophobic Black Iron Oxide | 0.37 |
| 3 | Titanium Dioxide/Methicone | 8.00 |
| 4 | Cyclopentasiloxane | 1.00 |
| 5 | Dow Corning 5225C | 12.00 |
| 5 | Simulated Vernix of Example 4 | 3.00 |
| 5 | Trimethylsiloxysilicate | 5.00 |
| 5 | Disteardimonium Hectorite | 1.00 |
| 5 | Tocopheryl Acetate | 0.10 |
| 6 | Dow Corning 556 Cosmetic Grade Fluid | 2.00 |

To make the composition described in Example 7, in the main kettle, the ingredients of Seq 1 and Seq 2 were added under propeller agitation at 75-78° C. In an auxiliary kettle, ingredients of Seq 3 were pre-mixed with propeller agitation. When the pigments were dispersed switch to homogenizer for 10-15 minutes. The ingredients of Seq 5 and Seq 6 were added into the main kettle at 75° C.-78° C. while mixing with propeller agitation, and the premixed Seq 3 ingredients were added under propeller agitation. The ingredient of Seq 4 were used to rinse the line that transferred Seq 3 into Seq 5-6. When the 10-15-minute mix was complete, switched to side-wiping agitation and cooled the batch to 30° C.

### Example 9: Moisturizing Long Wear Foundation Composition

A non-limiting example of skin care composition (a composition for moisturizing long wear foundation) including the simulated vernix composition of Example 4 was prepared. The skin care composition of Example 9 is a silicone free natural based foundation. The ingredients are listed below in Table 8:

TABLE 8
Moisturizing Long Wear Foundation Ingredients

| | | Weight |
|---|---|---|
| Seq | Ingredient | Percentage |
| 1 | Water | 57.81 |
| 1 | Magnesium Sulfate | 0.50 |
| 1 | Glyceryl Caprylate and Glyceryl Undecylenate | 0.50 |
| 2 | 1,3-Propanediol | 4.00 |
| 3 | Hydrogenated Farnesene | 11.00 |
| 3 | Ethylhexyl Hydroxystearate | 0.99 |
| 3 | Polyglyceryl-4 | 1.00 |
| | Diisostearate/Polyhydroxystearate/Sebacate | |
| 3 | Vitamin E Acetate | 0.20 |
| 3 | Simulated Vernix | 2.00 |
| 3 | Disteardimonium Hectorite | 2.00 |
| 3 | Hydrogenated Farnesene | 5.00 |
| 3 | Treated CI77499 | 0.10 |
| 3 | Treated CI77491 | 0.35 |
| 3 | Treated CI77492 | 1.35 |
| 3 | Treated Titanium Dioxide | 9.20 |
| 3 | Treated Mica | 3.00 |
| 3 | Lauryl Methacrylate and Glycol Dimethacrylate | 1.00 |
| | Crosspolymer | |

To make example 9, the materials of Seq 1 were added into the main kettle under propeller agitation (400-500 RPM) until uniform. In an auxiliary vessel, the materials of Seq 2 were premixed with propeller agitation. The Seq 2 material when then transferred into Seq 1 under propeller agitation for 10-15 minutes. Then, the batch was heated to 63° C.-65° C. under propeller agitation. In an auxiliary vessel, the material of Seq 3 were mixed with propeller agitation. When the pigments of Seq 3 were dispersed, the propeller agitation were switched to a homogenizer agitation (3000-3500 RPM) and the mixture were homogenized for 10-15 minutes and then added to main kettle. The batch were then cooled to 50° C.

Environmental pressures have been pushing formulators to remove silicones from cosmetic formulations. Since the formulation of Example 9 does not include silicone oil in the water emulsion, and still achieved desirable stability and was aesthetically pleasant.

### Example 10: Long Wear Lipstick Composition

A non-limiting example of skin care composition (a long wear lipstick composition) including the simulated vernix composition of Example 4 was prepared. The ingredients are listed below in Table 9:

TABLE 9
Long Wear Lipstick Ingredients

| Seq | Ingredients | Weight Percentage |
|---|---|---|
| 1 | Polyethylene wax | 8.00 |
| 1 | Candelilla wax | 3.00 |
| 1 | Simulated Vernix | 8.00 |
| 1 | Diglyceryl triisostearate | 15.00 |
| 1 | Macadamia nut oil fatty acid ester | 2.50 |
| 1 | Glyceryl tri-2-ethylhexanoate | 1.50 |
| 1 | Fine particle barium sulfate | 5.00 |
| 2 | Castor oil | 10.00 |
| 2 | Pigments | 5.00 |
| 2 | Camphor | 0.05 |
| 3 | MQ resin | 5.00 |
| 3 | Dimethiconol | 15.00 |
| 3 | Hydrogenated Farnesene | Qs |

To make the composition described in Example 10, the ingredients of Seq 2 were first mixed and grinded together with a roller mill, ball mill, or similar pigment grinding device. The ingredients in Seq 3 were mixed to form a homogeneous solution. The ingredients of Seq 1 are then added to a main kettle at 95° C. When Seq 1 ingredients formed a clear solution, Seq 2 was added to the main kettle while mixing. After the temperature is lowered to 75° C., Seq 3 ingredients were added to the kettle. The kettle was maintained at 75° C. until the entire bulk was uniform for pouring.

### Example 11: Skin Hydration Test

A non-limiting example of skin care composition (a moisturizing composition) was prepared. The ingredients are listed below in Table 10:

TABLE 10
Moisturizing Composition

| | | | Weight |
|---|---|---|---|
| Seq | Trade Name | Ingredients | Percentage |
| 1-10 | Neossance ™ Squalane | SQUALANE | 98.68 |
| 1-20 | Dermative Vernix | SQUALANE, PHYTOSTEROLS, | 0.5 |
| | | BUTYROSPERMUM PARKII | |
| | | (SHEA) BUTTER, OLEIC ACID, | |
| | | CERAMIDE NP, CANDELILLA | |
| | | CERA, BEHENYL ALCOHOL | |
| 1-30 | EVENING PRIME | OENOTHERA BIENNIS | 0.5 |
| | ROSE OIL | (EVENING PRIMROSE) OIL | |
| 1-40 | *Ronacare ® Bisabolol | BISABOLOL | 0.1 |
| 1-60 | Geranium Rosat | PELARGONIUM | 0.02 |
| | | GRAVEOLENS OIL | |
| 1-70 | *HYDRASALINOL | CAPRYLIC/CAPRIC | 0.2 |
| | | TRIGLYCERIDE, SALICORNIA | |
| | | HERBACEA EXTRACT | |

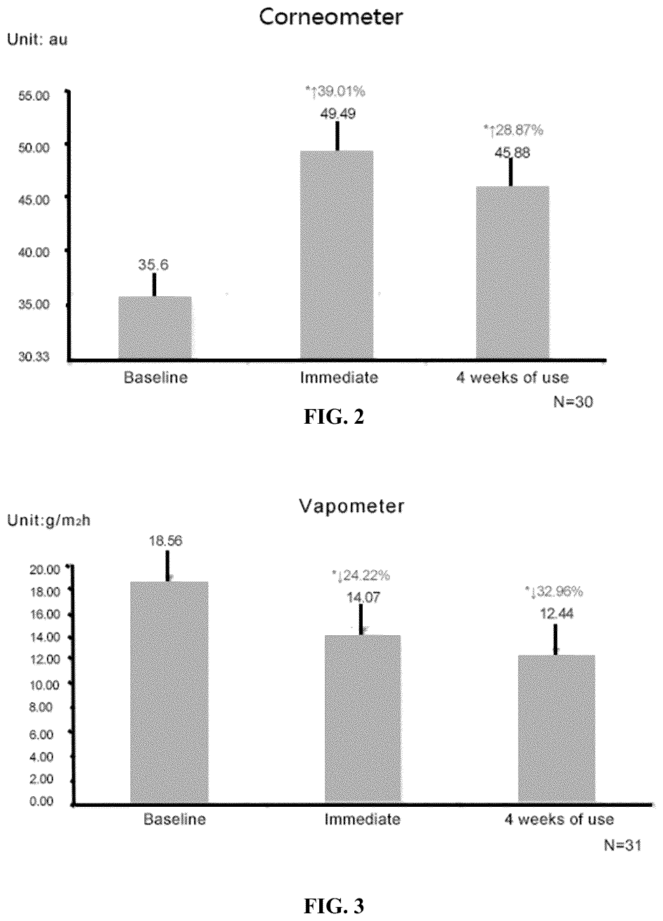

Results of the corneometer hydration test are presented in FIG. 2, and show an increase of approximately 39% immediately and approximately 29% with 4 weeks of use (N=30).

### Example 12: Transepidermal Water Loss (TEWL) Test

The effects of the skin care composition of Example 11 were measured in a transepidermal water loss (TEWL) test. Vapometer results of the TEWL test are presented in FIG. 3, and show a decrease in transdermal water loss (g/m²h) of approximately 24% immediately and a decrease of approximately 33% after 4 weeks (N=31).

### Example 13: Sensitive Skin Consumer Panel

A panel of consumers with sensitive skin tested the composition of Example 11 in a split face study, providing self-assessment of skin itchiness and irritation. Details of the study are as follows:
31 Volunteer meeting testing criteria conditions were recruited;
Initial visit, baseline measurements taken and skin surface washed with provided cleanser.
Skin allowed to equilibrate over 30 minutes in a controlled environment (20° C. and 50% humidity)
Facial images taken using VISIA-CR
Measurements taken using Corneometer and Vapometer
Clinician applied product and facial images were taken using VISIA-CR again after product absorption.
Measurements taken using Corneometer and Vapometer

Products given to panelists and advised on application parameters. Product was applied twice daily for four weeks.

After 2 weeks facial images were taken using VISIA-CR

After 4 weeks, facial skin was washed using provided cleanser.

Skin was allowed to equilibrate for 30 minutes prior to image capturing with VISIA-CR

Measurements taken with Corneometer and Vapometer

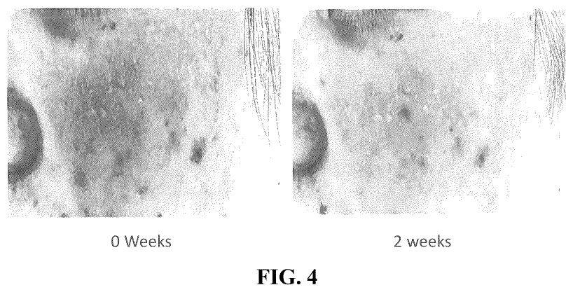

A sample image showing decreased redness and irritation after 2 weeks of use is shown in FIG. 4. A summary of self-assessment results is presented in Table 13:

TABLE 13
Sensitive Skin Consumer Panel

| | Placebo | Vernix Formulation |
|---|---|---|
| Very Effective | 1 | 4 |
| Effective | 5 | 9 |
| Somewhat | 6 | 3 |
| Effective | | |
| Not Effective | 6 | 2 |

More than twice as many panelists found the vernix formulation to be effective or very effective at reducing skin itchiness or irritation compared to the placebo. 83% found the Vernix formulation to be effective or very effective at reducing skin itchiness and irritation compared to 33% of the placebo.

### ENUMERATED EMBODIMENTS

In some aspects, the present invention is directed to the following non-limiting embodiments:
Embodiment 1: A composition comprising: a squalane compound, wherein the amount of the squalane compound ranges from 50 parts by weight to 95 parts by weight based on 100 parts by weight of the composition; a liquid monoester, wherein the amount of the liquid monoester ranges from 5 parts by weight to 30 parts by weight based on 100 parts by weight of the composition; and a vernix lipid, which comprises at least one selected from the group consisting of a cholesterol ester, a ceramide, a cholesterol, a phospholipid, a wax diester, a glycosphingolipid, and a cholesterol sulfate, and wherein the amount of the vernix lipid ranges from 0.0.05 parts by weight to 20 parts by weight based on 100 parts by weight of the composition, wherein the composition is a uniform oil phase mixture.
Embodiment 2: The composition of Embodiment 1, wherein the vernix lipid comprises a ceramide.
Embodiment 3: The composition of Embodiment 2, wherein the ceramide comprises at least one selected from the group consisting of ceramide II and ceramide III.
Embodiment 4: The composition of Embodiment 2 or 3, wherein the amount of the ceramide ranges from 0.05 parts by weight to about 10 parts by weight based on 100 parts by weight of the composition.
Embodiment 5: The composition of any one of Embodiments 1-4, wherein the vernix lipid further comprises at least one selected from the group consisting of a cholesterol ester, a cholesterol, a phospholipid, a wax diester, a glycosphingolipid, and a cholesterol sulfate.
Embodiment 6: The composition of any one of Embodiments 1-5, wherein the squalane compound comprises squalane.
Embodiment 7: The composition of any one of Embodiments 1-6, wherein the liquid monoester comprises at least one selected from the group consisting of jojoba oil and a polyglyceryl isostearate.
Embodiment 8: The composition of any one of Embodiments 1-7, which is at least one selected from the group consisting of a cosmetic composition, a skin care composition, a personal care composition, and a pharmaceutical composition.
Embodiment 9: A method of preparing the composition of any one of any one of Embodiments 1-8, the method comprising: heating the squalane to an elevated temperature; adding the liquid monoester and the vernix lipid to the heated squalane to form a mixture while maintaining the elevated temperature; and cooling down the mixture to room temperature.
Embodiment 10: An emulsion, comprising: the composition of any one of Embodiments 1-8; and water.
Embodiment 11: The emulsion of Embodiment 10, further comprising an emulsifier.
Embodiment 12: The emulsion of any one of Embodiments 10-11, which is an oil-in-water emulsion or a water-in-oil emulsion.
Embodiment 13: The emulsion of any one of Embodiments 10-12, which is at least one selected from the group consisting of a cosmetic composition, a skin care composition, a personal care composition, and a pharmaceutical composition.
Embodiment 14: A composition comprising: a squalane compound, wherein the amount of the squalane compound ranges from 50 parts by weight to 95 parts by weight based on 100 parts by weight of the composition; a triglyceride, wherein the amount of the triglyceride ranges from 5 parts by weight to 30 parts by weight based on 100 parts by weight of the composition; and a vernix lipid, which comprises at least one selected from the group consisting of a cholesterol ester, a ceramide, a cholesterol, a phospholipid, a wax diester, a glycosphingolipid, and a cholesterol sulfate, and wherein the amount of the vernix lipid ranges from 0.05 parts by weight to 20 parts by weight based on 100 parts by weight of the composition, wherein the composition is a uniform oil phase mixture.
Embodiment 15: The composition of Embodiment 14, wherein the vernix lipid comprises a ceramide.
Embodiment 16: The composition of Embodiment 15, wherein the ceramide comprises at least one selected from the group consisting of ceramide II and ceramide III.
Embodiment 17: The composition of Embodiment 15 or 16, wherein the amount of the ceramide ranges from 0.05 parts by weight to about 10 parts by weight based on 100 parts by weight of the composition.
Embodiment 18: The composition of any one of Embodiments 14-17, wherein the vernix lipid further comprises at least one selected from the group consisting of a cholesterol ester, a cholesterol, a phospholipid, a wax diester, a glycosphingolipid, and a cholesterol sulfate.
Embodiment 19: The composition of any one of Embodiments 14-18, wherein the squalane compound comprises squalane.
Embodiment 20: The composition of any one of Embodiments 14-19, wherein the triglyceride includes shea butter.
Embodiment 21: The composition of any one of Embodiments 14-20, further comprising at least one of a fatty acid and a fatty alcohol.
Embodiment 22: The composition of Embodiment 21, wherein the fatty acid is oleic acid and the fatty alcohol is behenyl alcohol.
Embodiment 23: The composition of any one of Embodiments 14-22, which is at least one selected from the group consisting of a cosmetic composition, a skin care composition, a personal care composition, and a pharmaceutical composition.
Embodiment 24: A method of supplementing naturally occurring lipids on the skin, comprising applying to the skin a composition according to any one of Embodiments 1-8, thereby forming a simulated vernix on the skin.
Embodiment 25: A method of supplementing naturally occurring lipids on the skin, comprising applying to the skin a composition according to any one of Embodiments 14-23, thereby forming a simulated vernix on the skin.

The foregoing outlines features of several embodiments so that those skilled in the art may better understand the aspects of the present disclosure. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions, and alterations herein without departing from the spirit and scope of the present disclosure.

What is claimed is:

1. A composition comprising:
a squalane compound, wherein the amount of the squalane compound ranges from 50 parts by weight to 95 parts by weight based on 100 parts by weight of the composition;
a liquid monoester, wherein the amount of the liquid monoester ranges from 5 parts by weight to 30 parts by weight based on 100 parts by weight of the composition; and
a vernix lipid, which comprises at least one selected from the group consisting of a cholesterol ester, a ceramide, a cholesterol, a phospholipid, a wax diester, a glycosphingolipid, and a cholesterol sulfate, and wherein the amount of the vernix lipid ranges from 0.0.05 parts by weight to 20 parts by weight based on 100 parts by weight of the composition,
wherein the composition is a uniform oil phase mixture.

2. The composition of claim 1, wherein the vernix lipid comprises a ceramide.

3. The composition of claim 2, wherein the ceramide comprises at least one selected from the group consisting of ceramide II and ceramide III.

4. The composition of claim 2, wherein the amount of the ceramide ranges from 0.05 parts by weight to about 10 parts by weight based on 100 parts by weight of the composition.

5. The composition of claim 2, wherein the vernix lipid further comprises at least one selected from the group consisting of a cholesterol ester, a cholesterol, a phospholipid, a wax diester, a glycosphingolipid, and a cholesterol sulfate.

6. The composition of claim 1, wherein the squalane compound comprises squalane.

7. The composition of claim 1, wherein the liquid monoester comprises at least one selected from the group consisting of jojoba oil and a polyglyceryl isostearate.

8. The composition of claim 1, which is at least one selected from the group consisting of a cosmetic composition, a skin care composition, a personal care composition, and a pharmaceutical composition.

9. A method of preparing the composition of claim 1, the method comprising:
heating the squalane to an elevated temperature;
adding the liquid monoester and the vernix lipid to the heated squalane to form a mixture while maintaining the elevated temperature; and
cooling down the mixture to room temperature.

10. An emulsion, comprising:
the composition of claim 1; and
water.

11. The emulsion of claim 10, further comprising an emulsifier.

12. The emulsion of claim 10, which is an oil-in-water emulsion or a water-in-oil emulsion.

13. The emulsion of claim 10, which is at least one selected from the group consisting of a cosmetic composition, a skin care composition, a personal care composition, and a pharmaceutical composition.

14. A composition comprising:
a squalane compound, wherein the amount of the squalane compound ranges from 50 parts by weight to 95 parts by weight based on 100 parts by weight of the composition;
a triglyceride, wherein the amount of the triglyceride ranges from 5 parts by weight to 30 parts by weight based on 100 parts by weight of the composition; and
a vernix lipid, which comprises at least one selected from the group consisting of a cholesterol ester, a ceramide, a cholesterol, a phospholipid, a wax diester, a glycosphingolipid, and a cholesterol sulfate, and wherein the amount of the vernix lipid ranges from 0.05 parts by weight to 20 parts by weight based on 100 parts by weight of the composition,
wherein the composition is a uniform oil phase mixture.

15. The composition of claim 14, wherein the vernix lipid comprises a ceramide.

16. The composition of claim 15, wherein the ceramide comprises at least one selected from the group consisting of ceramide II and ceramide III.

17. The composition of claim 15, wherein the amount of the ceramide ranges from 0.05 parts by weight to about 10 parts by weight based on 100 parts by weight of the composition.

18. The composition of claim 14, wherein the vernix lipid further comprises at least one selected from the group consisting of a cholesterol ester, a cholesterol, a phospholipid, a wax diester, a glycosphingolipid, and a cholesterol sulfate.

19. The composition of claim 14, wherein the squalane compound comprises squalane.

20. The composition of claim 14, wherein the triglyceride includes shea butter.

21. The composition of claim 14, further comprising at least one of a fatty acid and a fatty alcohol.

22. The composition of claim 21, wherein the fatty acid is oleic acid and the fatty alcohol is behenyl alcohol.

23. The composition of claim 14, which is at least one selected from the group consisting of a cosmetic composition, a skin care composition, a personal care composition, and a pharmaceutical composition.

24. A method of supplementing naturally occurring lipids on the skin, comprising applying to the skin a composition according to claim 1, thereby forming a simulated vernix on the skin.

25. A method of supplementing naturally occurring lipids on the skin, comprising applying to the skin a composition according to claim 14, thereby forming a simulated vernix on the skin.
